(19) United States
(12) Patent Application Publication
(10) Pub. No.: US 2025/0387157 A1
(43) Pub. Date: Dec. 25, 2025

(54) TISSUE ABLATION DEVICE AND SYSTEM

(71) Applicant: Matan SHABAT, Eilat (IL)

(72) Inventor: Matan SHABAT, Eilat (IL)

(21) Appl. No.: 18/879,781

(22) PCT Filed: Jun. 28, 2023
(86) PCT No.: PCT/IL2023/050673

§ 371 Date: Dec. 29, 2024

(30) Foreign Application Priority Data
Jun. 28, 2022 (IL) 294383

(51) Int. Cl. A61B 18/14; A61B 17/00; A61B 18/00

(52) U.S. Cl. CPC A61B 18/1492; A61B 2017/00402; A61B 2018/00267; A61B 2018/00559; A61B 2018/00577; A61B 2218/002; A61B 2218/007

(57) ABSTRACT

A tissue ablation device and system and in particular, to such a device and system providing improved ablation and removal of gynecological growths.

### FIELD OF THE INVENTION

The present invention relates to a tissue ablation device and system and in particular, to such a device and system providing improved ablation and removal of gynecological growths.

### BACKGROUND OF THE INVENTION

Endometriosis is a condition afflicting women of child-bearing age which is characterized by the growth of endometrial tissue in areas outside the uterus. These extrauterine endometrial growths are a leading cause of pelvic pain and can also cause infertility.

As is the case with the uterine lining, extrauterine endometrial growths typically respond to the varying levels of estrogen associated with the menstrual cycle. Thus, endometrial growths proceed through a cycle of proliferation and breakdown. Unlike the uterine lining, however, the body is unable to shed the extrauterine endometrial growths, and breakdown of this tissue results in internal bleeding, inflammation of the surrounding area and formation of scar tissue. A number of complications can also arise, including rupture of growths, which can spread the growths to new regions of the body, and the formation of adhesions.

Endometriosis is usually confined to the peritoneal or serosal surfaces of abdominal organs, commonly the ovaries, posterior broad ligament, posterior cul-de-sac, and uterosacral ligaments (sometimes forming uterosacral nodules). Less common sites include the serosal surfaces of the small and large bowel, ureters, bladder, vagina, surgical scars, pleura, and pericardium.

### SUMMARY OF THE INVENTION

The present invention overcomes the deficiencies of the background art by providing a device and system for localized tissue ablation and particularly configured for removal of gynecological growths such as endometrioma.

Embodiments of the present invention provide a gynecological device and system configured for the removal of cysts or the like growths, for example endometrioma.

Embodiments of the present invention provided a device and system for a tissue ablation device, the device comprising, a body, a working channel module featuring at least one working channel, fluid flow module, an electronics module, a sensor module, and an expandible ablation member that is functionally coupled to an actuator. Most preferably, the tissue ablation member and actuator are configured to be coupled so as to allow the ablation member to rotate when in use.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic block diagrams of an exemplary device and system according to embodiments of the present invention;

FIG. 2 is a schematic illustrative diagram of an exemplary device according to an embodiment of the present invention;

FIG. 3 is a schematic illustrative diagram of an exemplary device according to an embodiment of the present invention.

FIG. 4 is a schematic illustrative diagram of an exemplary device according to an embodiment of the present invention;

FIG. 5 is a schematic illustrative diagram of an exemplary device and system according to an embodiment of the present invention; and

FIG. 6 is a schematic illustrative diagram of an exemplary device and system according to an embodiment of the present invention.

### DESCRIPTION OF THE PREFERRED EMBODIMENTS

The principles and operation of the present invention may be better understood with reference to the drawings and the accompanying description. The following figure reference labels are used throughout the description to refer to similarly functioning components are used throughout the specification hereinbelow.
50 auxiliary devices;
10 device introducer;
100 tissue ablation device;
102 device body;
102c coupling members;
102d distal portion;
102p proximal portion;
104 fluid flow module;
104a negative pressure sub-module;
104b positive pressure sub-module;
104c flow module controller;
104e auxiliary and/or external flow module;
105 expandible ablation/pulverizing member;
105s cutting surface;
106 working channel module;
106a,b working channels;
106i introducing port;
108 actuator;
110 electronics module;
111 user interface (UI);
112 power module;
114 controller and/or processor module;
116 communication module;
118 memory module;
120 sensor module;
121 pressure sensor;
122 temperature sensor;
124 pH sensor;
125 positional sensor;
126 imaging sensor;
128 flow sensor;
150 system;

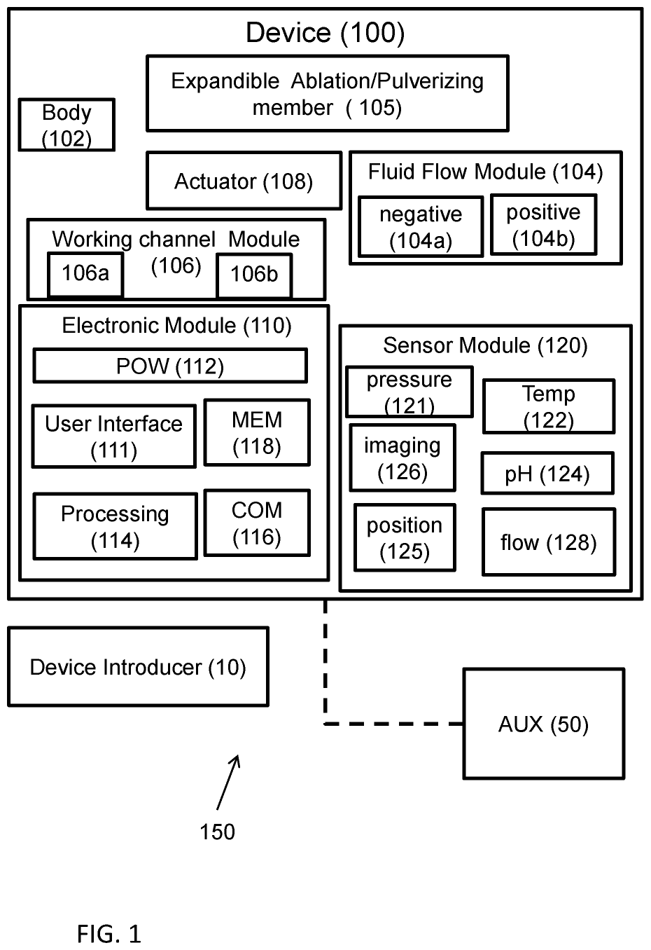

FIG. 1 shows a schematic block diagram of an tissue ablation device 100 and a system 150 thereof, according to embodiments of the present invention. Tissue ablation device 100 is preferably utilized to treat and/or drain and/or remove and/or ablate cysts, growths as a result of endometriosis, for example including but not limited to endometrioma.

In particular device 100 may be configured to drain and/or pulverize and/or ablate such endometriosis growths and/or endometrioma and/or endometrioma known as “chocolate endometrioma” and/or cysts, with an expandible ablation and/or pulverizing member 105 in order to remove such tissue.

In some embodiments device 100 may be used to facilitate drainage of an endometrioma and/or a chocolate endometrioma wherein the pulverizing member 105 may be utilized to facilitate mechanical breakdown of the coagulated tissue matter defining and/or internal to the endometrioma lumen, in particular the chocolate endometrioma, so as facilitate its drainage and/or removal by way of aspiration and/or suctioning.

Tissue ablation device 100 comprises a controllably expandible ablation and/or pulverizing member 105 that is configured ablate and/or pulverize a targeted tissue site and/or treatment site, for example a cyst, growth such as an endometrioma or the like endometriosis growth.

In embodiments, expandible member 105 is preferably configured to undertake the ablating and/or pulverizing action, optionally and preferably by way of rotation, wherein the rotation is actuated with an actuator 108. In embodiments rotation of ablating and/or pulverizing member 105 may be configured to rotate about a long axis of device 100.

In embodiments, the rotational parameters of member 105 may be controllable with actuator 108 and electronics module 110. In embodiments, the rotational parameters of the ablation member 105 may be selected based on at least one or more parameters for example including but not limited to torque, resistance, speed, acceleration, power, velocity, number of revolutions any combination thereof or the like. In embodiments, the rotational parameters of the ablation member 105 may be selected based on the treatment site and/or tissue type and/or the like tissue parameters, for example including but not limited to tissue stiffness, tissue hardness, treatment site viscosity, mass viscosity, tissue density, tissue type, malleability, tissue resistance, tissue malleability, the like tissue parameters or any combination thereof.

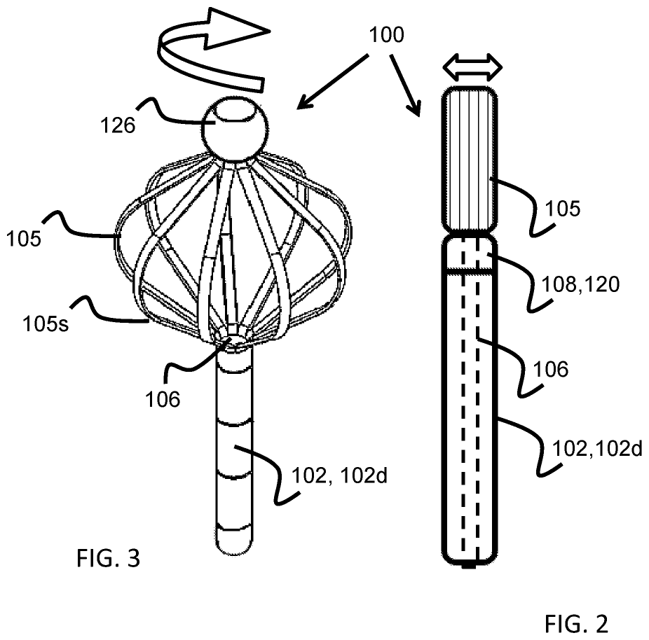

In embodiments, expandable ablation member 105 is preferably provided in a small profile configuration when introduced to the anatomy until it is delivered at the tissue treatment site and/or ablation site, a non-limiting schematic depiction of which is shown in FIG. 2.

In some embodiments, the small profile configuration of member 105 may be introduced internal to an endometrioma lumen and expanded internal to the endometrioma cyst and/or lesion. Once expanded, member 105 may be utilized to pulverize the tissue and/or matter internal to the endometrioma lumen.

In embodiments, an expanded open state configuration of member 105, a non-limiting schematic depiction example of which is shown in FIG. 3, is configured to be utilized when ablating and/or pulverizing the targeted tissue site and/or growth at the target tissue site. Most preferably, the shape and/or size and/or volume and/or at least one dimension of the expanded configuration is controllable and may be adjusted. In embodiments, the dimension of the expanded configuration of member 105 may be based on optional parameters for example including but not limited to the size of the targeted tissue site and/or growth and/or cyst and/or endometrioma, or the like. For example, a large cyst and/or growth would require a larger pulverizing member 105 while a smaller cyst and/or growth would require an appropriately sized pulverizing member 105.

In embodiments, at least one dimension of the pulverizing member 105 may be controlled for example including but not limited to diameter, length, width, volume, height, the like or any combination thereof.

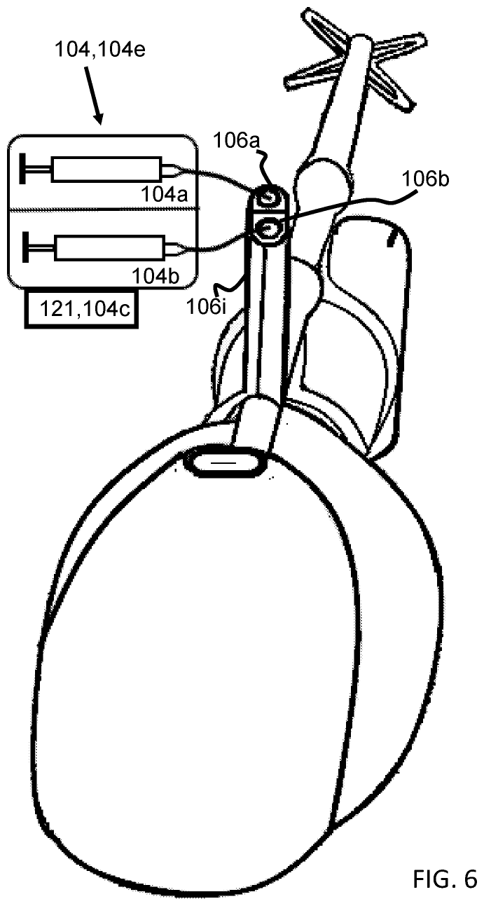

In embodiments, the expanded state of ablation and/or pulverizing member 105 is controllable based on ascertaining the ablation site, while disposed at the ablation site. Optionally, ascertaining the ablation site may be preferably provided with at least one or more sensor of sensor module 120, for example image sensor 126 or the like sensors. Optionally, imaging of the treatment site utilizing imaging sensors, such as an ultrasound transducer, may be provided with an external auxiliary imaging device 50 that is associated with device 100 forming system 150, for example as shown in FIG. 5.

In embodiment expandible member 105 may comprise at least one or more cutting surfaces and or edges 105s, a non-limiting example of which is depicted in FIG. 3, and FIG. 5. In optional embodiments the cutting surfaces may be exposed in the expanded state.

In embodiment when assuming the expanded state expandible member 105 may comprise at least two or more cutting surfaces. Optionally, a first cutting surface may have a first orientation, and a second cutting surface may have a second, different, orientation. For example, a first cutting surface orientation may be horizontal and a second cutting surface orientation may be vertical, or vice versa, or a combination thereof. In embodiments, the plurality of cutting surfaces may be disposed along an end of member 105 and/or along the length of member 105, or any combination thereof.

In embodiments pulverizing and/or ablating member 105 is functionally associated with an actuator 108, provided to mobilize and/or render member 105 functional. In embodiments, actuator 108 may be provided in optional form for example including but not limited to a piezoelectric actuator, MEMS motor, a motor, magnetic actuator, the like actuator. In embodiments, actuator 108 may be disposed along any portion of device 100, most preferably about the body 102 of device 100, for example in a proximal portion 102p or a distal portion 102d of body 102. Optionally actuator 108 may be directly associated with member 105, for example as shown in FIG. 2.

In an optional embodiment, actuator 108 may be remotely but functionally associated with member 105. For example, member 105 disposed at a distal portion of device 100 while actuator 108 may be disposed proximal portion of device 100.

In embodiments actuator 108 provides for rotating member 105 about a longitudinal axis of device 100, for example as schematically illustrated by directional arrows in FIG. 3.

Tissue ablation device 100 comprises a housing and/or body 102 that is configured to be a housing of at least a portion of device 100. In some embodiments at least a portion of body 102 may be shaped and configured in the form of a handle to be manually operated by a practitioner, along at least a proximal portion 102p thereof.

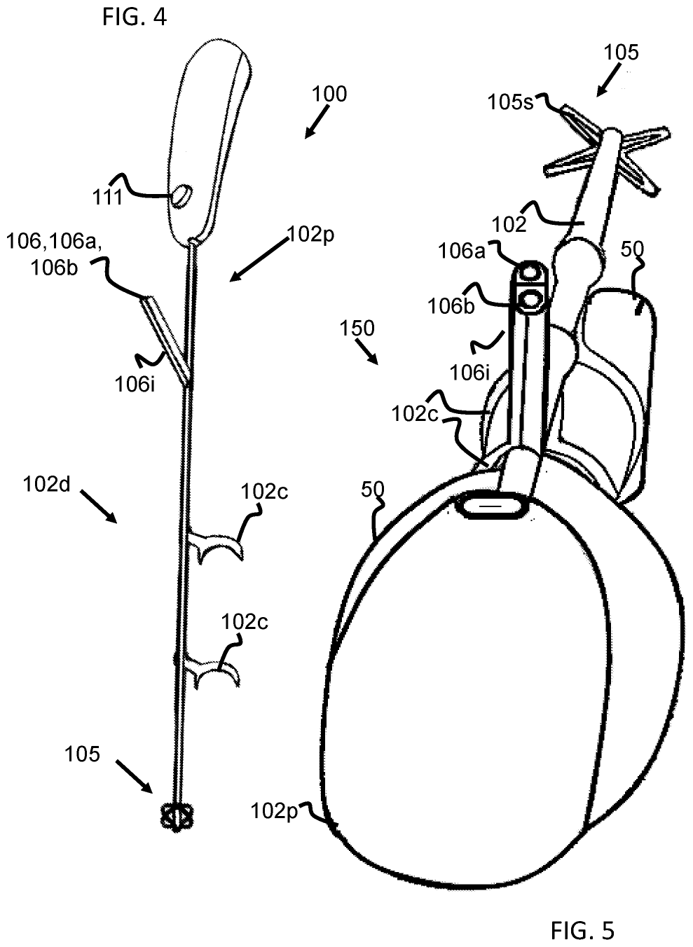

In embodiments as best shown in FIG. 4-5, body portion 102 features a proximal portion 102p and a distal portion 102d. In embodiment proximal portion 102p featuring a handle portion for facilitating manipulation and operation of device 100 and/or system 150. In embodiments the distal portion 102d featuring an elongated body and/or shaft 102s featuring the working channel(s) 106, 106a, 106b, and expandable member 105. Preferably shaft 102s comprises an access port 106i, so as to allow access to the working channels 106 during use. Preferably, access port 106i may be configured as a multi-lumen port, as best seen in FIG. 4-5, featuring at least two lumens corresponding to the number of working channels 106 as to allow access to individual working channels during use.

In some embodiments, a distal end of body portion 102d may further comprise a multi-lumen needle that is preferably associated with at least one or more working channels 106, 106a, 106b.

In embodiments, the length of body distal portion 102d may be configured to have an adjustable length. In an optional embodiment, distal portion 102d may be configured to be telescopic.

In embodiments, at least a portion of distal body portion 102d may be configured to be maneuverable and/or positionally adjustable such that it is maneuverable and/or bendable with the anatomy, for example similar to a guidewire, to properly delivery and/or position device 100 relative to the treatment site and in particular the ablation member 105 relative to the treatment site.

In some embodiments at least a portion of body 102 may be configured and/or shaped to accommodate and/or compliment additional tools and/or devices to facilitate use thereof. For example, a portion of body 102 may be configured to associate with and introducing device 10 for example a gynecological tools to facilitate placement within the uterus, for example including but not limited to speculum, trocar, sheath, sleeve or the like. For example, a portion of body 102 may be configured to be associated with an auxiliary device 50 for example an imaging device in the form of an ultrasound probe and/or an intravaginal ultrasound probe, for example as shown in FIG. 4-5.

In some embodiments body 102 may be configured to feature coupling members 102c, FIG. 4-5, preferably for facilitating coupling of at least one or more auxiliary devices 50 and/or introducing device 10.

In some embodiments body 102 may be configured to be associated with and/or operated by an automated introduction devices 10 for example including but not limited to a robotic arms or the like advancing device. For example, body 102 may be configured to associate with a robotic arm or the like motorized advancing tool so as to facilitate advancing at least a portion of device 100 toward a targeted tissue ablation site.

In embodiments device 100 optionally and preferably comprises a fluid flow module 104 configured to both receive and/or deliver a fluid sample to and/or from the targeted tissue ablation site. In embodiments fluid flow module 104 may be configured to receive a fluid sample, for example a medicament and/or tissue sample, by way of suctioning and/or up-taking a fluid from the treatment site and/or a receptacle, in preparation for application to a targeted tissue ablation site.

In embodiments fluid flow module 104 may therefore comprise at least one or both of a negative pressure sub-module 104a, configured for generating suctioning and/or vacuum to facilitate fluid uptake to and/or from a tissue ablation site, and/or a positive pressure sub-module 104b configured for ejecting and/or delivering a fluid sample to the tissue ablation site.

In embodiments fluid flow module 104, 104a, 104b may comprise but is not limited to at least one or more of: a motors, hydraulic motor, electromagnetic motor, piston, air piston, spring loaded movement, gear, linear spring, windings, electromagnet, pump, compressor, the like or any combination thereof.

In some embodiments, fluid flow module 104 may be utilized to drain a chocolate endometrioma by introducing a medicament, for example via positive pressure sub-module 104b, to the internal lumen of the endometrioma. The medicament configured to dissolve and/or break down coagulated tissue found within the endometrioma lumen. Following the introduction of the medicament, ablation member 105 may be introduced into the endometrioma lumen to internally pulverize the tissue internal to the endometrioma lumen by activating actuator 108 and rotating member 105. Once pulverized, the internal tissue may be suctioned and/or aspirated with negative pressure sub-module 104a, wherein the suctioned tissue is evacuated and/or drained from the endometrioma tissue site optionally and preferably via at least one or more working channel 106.

In embodiments device 100 comprises a working channel module 106 having at least one or more working channels 106a,b to allow for introducing additional tools and/or fluid to facilitate undertaking tissue treatment at the treatment site wherein an endometrioma may be ablated and/or drained. For example, such a working channel may be used to introduce a medicament, a suction tube, light source, heat source, tools, cauterizing tool, laser therapy tool, or the like additional tools or the like at or near the tissue treatment site, most preferably an endometrioma. In embodiments each working channel 106a of channel module 106 may be configured as an elongated hollow tube that may directly uptake and/or receive additional tools and/or fluids.

In some embodiments some working channels may be dedicated as tool channels while others may be dedicated fluid handling channels.

In some embodiments two working channels 106a,b may be a configured as a fluid channel in opposite directions a first channel, 106a, for introducing a flowing fluid and a second channel, 106b, for removing and/or aspirating a flowing fluid.

In some embodiments an individual working channels 106a,b may be configured as a dual function channel acting as both a fluid channel and working tool channel, for example a sheath.

In some embodiments channels 106a,b may be configured in the form of a sheath and/or guiding sheath for receiving a catheter.

In embodiments at least a portion of working channel module 106 may feature and/or be associated with at least one or more sensors of sensor module 120.

In embodiments working channel(s) 106,106a,106b may be configured to feature an introduction port 106i, FIG. 4-5, preferably disposed along a shaft portion of body 102 preferably a proximal portion of body so as to allow access to the working channels during use.

In some embodiments introduction port 106i may be provided in the form of a multi-lumen access point and/or port, for example as shown in FIG. 4-5.

In some embodiments, a distal end of at least one or more working channel 106,106a,106b may be configured to transcend member 105.

In some embodiments, a distal end of at least one or more working channels 106,106a, 106b may be configured to align with a proximal end of ablation member 105, for example as shown in FIG. 3.

In embodiments tissue ablation device 100 may comprise electronic module 110 comprising electronics circuitry, hardware, software to render device 100 functional and/or operational.

In embodiments electronics module 110 may comprise a plurality of optional sub-modules for example including but not limited to a power supply module 112, controller and/or processor module 114, user interface module 111, and memory module 118. In a preferred embodiments electronics module 110 may further comprise a communication module 116.

In embodiments electronics module 110 may be functionally associated with and/or comprise a sensor module 120.

In embodiments, User Interface (UI) module 111 may provide a user with means for interfacing with device 100 preferably via processor module 114. User interface 111 may be provided in the form of an activation button, audiovisual display, the like or any combination thereof. In embodiments, UI module 111 may be provided in optional forms for example including but not limited to buttons, dials, displays, alphanumeric display, touch screen, touch pad, buzzer, tactile pad, at least one light emitting diode (LED), at least one organic LED (OLED), speakers, microphone, or any combination thereof.

In embodiments processor module 114 provides the necessary processing hardware and/or software necessary to render device 100 functional. In embodiments controller and/or processor module 114 may provide for controlling any portion of device 100 and in particular fluid flow module 104 and sensor module 120.

In embodiments power module 112 provides the necessary hardware and/or software to power device 100 therein rendering device 100 operational. Power module 112 may for example be provided in optional forms for example including but not limited to battery, rechargeable induction battery, induction coil, capacitors, super capacitors, inductors the like power source or any combination thereof.

In embodiments communication module 116 preferably provides the necessary hardware and/or software to facilitate communication for device 100 and in particular with auxiliary devices 50 and/or introducing device 10 forming system 150. Optionally communication module 116 may be utilized to communicate with an optional auxiliary devices 50. For example, an auxiliary device 50 may for example include but is not limited to a smartphone, mobile processing and communication device, imaging device, server, computer, healthcare service provider dedicated system, first respondent call center, health care call center, the like or any combination thereof.

In embodiments auxiliary device 50 may be provided in the form of an ultrasound imaging scanning device, for example in the form of a transvaginal ultrasound scanning transducer and/or probe and/or device as depicted in FIG. 5.

In some embodiments communications module 116 may be utilize various communication protocols for example including but not limited to wireless communication, cellular communication, wired communication, near field communication, BLUETOOTH, ZIGBEE, optical communication, the like and/or any combination thereof.

In embodiments memory module 118 provides the necessary hardware and/or software to facilitate operations of device 100 by enabling storing and/or retrieving stored data and/or the like as is known in the art.

In embodiments sensor module 120 provides the necessary hardware and/or software to facilitate operations of at least one or more sensor(s) associated with device 100 to enable sensing various events in and around device 100 both internal and/or external to device 100. In some embodiments, for example, sensor module 120 may be configured to sense the environment external to device 100, for example, the tissue ablation site. For example, sensor module 120 may be utilized to provide imaging of the tissue ablation site, measuring the temperature, measuring the pH, tissue viscosity, fluid viscosity, tissue site parameters, or the like conditions in and around the tissue ablation site.

In embodiments sensor module 120 may comprise at least one or more sensor selected from the group consisting of: image sensor 126, temperature sensor 122, pH sensor124, flow-meter 128, pulse oximeter, pressure sensor 121, acoustic sensor, microphone, positional sensor 125, infrared sensor, optical sensor, wavelength specific imaging sensor, the like or any combination thereof.

In embodiments sensor module 120 may be disposed along any portion of device 100, and in particular near the distal and/or working end of body 102. In embodiments, at least some sensors of sensor module 120 may be disposed adjacent to ablation member 105.

In embodiments image sensor 126 may be provided in optional forms, for example including but not limited to camera. In some embodiments image sensor may be provided in the form of ultrasound sensor, that may optionally be disposed along a portion of body 102. In some embodiments image sensor 126 may be provided in the form of wavelength specific imaging device such as an infrared optical sensor and infrared light source.

In embodiments an image sensor 126, optionally in the form of a camera, may be configured to provide image processing and/or analysis so as to infer and/or obtain parametric data relating to the tissue ablation site and/or the suspect targeted tissue site. For example, such image processing analysis may provide to determine at least one parameter associated with the targeted tissue ablation site, such as a growth and/or cyst for example including but not limited to: length, width, height, area, volume, shape, surface area, tissue type, or any combination thereof.

In some embodiments sensor module 120 may comprise at least one position sensor 125 configured to identify the position a sample channel 106 so as to convey the spatial location of a targeted tissue ablation site. In embodiments position sensor 125 may be provided in optional forms for example including but not limited to optical sensors, mechanical sensors, electromagnetic sensor, induction sensor, magnetic based sensors or the like sensor provided for determining and the relative position of device 100 within the human anatomy.

In embodiments, device 100 may be used to define a system 150,comprising tissue ablation device 100 that is in communication with a at least one or more auxiliary device 50. In embodiments auxiliary device 50 may be provided in optional forms for example including but not limited to a server, computer, smartphone, or the like processing and communication device capable of receiving and/or exchanging data from device 100 so as to process the data. In embodiments, auxiliary device 50 is preferably utilized to apply big data algorithms and/or the like artificial intelligence algorithms to analyze data received from device 100. Optionally communication between auxiliary device 50 and device 100 is facilitated with communication module 116 utilizing any communication protocol for example including wireless, cellular, wired, near field, the like or any combination thereof.

In embodiments system 150 and/or device 100 and/or auxiliary device 50 may communicate with additional auxiliary devices to undertake processing of data associated with system 150 and/or device 100. For example, such auxiliary device communication devices may for example include but is not limited to a smartphone, mobile processing and communication device, imaging device, server, computer, healthcare service provider dedicated system, first respondent call center, health care call center, the like or any combination thereof.

FIG. 2 shows a schematic illustrative diagram of a non-limiting example of a configuration of device 100 showing ablation and/or pulverizing member 105 in the delivery configuration wherein a minimal profile is assumed. Once reaching the targeted tissue site member 105 is preferably expanded, for example as schematically shown with directional arrows.

In embodiments, once at the tissue treatment site and/or delivery site sensor module 120 may be utilized to ascertain the size of the tissue to be treated and/or ablated, and member 105 is expanded to assume a shape proportional to the tissue site to render the ablation process more efficient.

In embodiments, the expansion of member 105 to a selected diameter and/or size of member 105 is controllable.

FIG. 3 shows a schematic illustrative diagram of a non-limiting example of a configuration of device 100, wherein the ablating and/or pulverizing member 105 is depicted in an optional expanded stated. In embodiments when in the expanded state reveals at least one or more cutting surfaces 105s, a non-limiting example of which is depicted.

In embodiments when in the expanded state, actuator 108 is configured to optionally rotate member 105, for example as shown with the direction arrow, so as to actuate the cutting surface around the tissue ablation site.

Preferably, during ablation for example by way of rotation of member 105 at least one or more working channels of channel module 106 is configured to aspirate and/or suction tissue as it is pulverized and/or ablated away from the tissue site.

In embodiments, an image sensor 126 may be utilized to visualize and communicate images of the process in real time. Optionally and preferably an image sensor may optionally further provide for assessing parameters relating to the tissue site so as to determine the size and/or volume degree of expansion required of member 105.

In embodiments member 105 may be configured to be a single use device.

In embodiments device 100 or portions thereof may be configured to be a single use device.

In embodiments, preferably following the ablation process, member 105 is compressed to reassume the small and/or delivery configuration, for example as shown in FIG. 2, so as to be removed from the tissue delivery site.

In embodiments member 105 is preferably configured to be provided from optional biocompatible materials for example including but not limited to polymers, alloys, smart materials, shape memory materials, nitinol, materials exhibiting plastic deformation, super-elastic metal alloy which transforms from an austenitic state to a martensitic state, any combination thereof or the like as is known in the art.

Now collectively referring to FIG. 4-5 that show a perspective view of a non-limiting configuration of device 100 as previously described showing an expanded ablation member 105 disposed a distal end of distal portion 102d. The device is utilized to drain and/or remove tissue from a remote tissue treatment site, for example and endometrioma, wherein optional fluids and/or devices may be introduced to a working channel 106 via introduction port 106i have a multiple lumen 106a, 106b. Body 102 further comprises coupling member 102c disposed along the length of body 102 so as to couple optional auxiliary device 50, for example an imaging ultrasound transduces as shown in FIG. 5.

In embodiments, fluid flow module 104 may be provided in the form of a two syringe assembly and associated via introduction port 106i where a first syringe utilized, acting as a negative pressure sub-module 104a, utilized to aspirate and/or suction the tissue treatment site, and a second syringe utilized to act as a positive pressure sub-module 104b so as to introduce a fluid to the treatment site, for example a saline solution. Most preferably device 100 and in particular sensor module 120 features a pressure sensor to measure and equilibrate pressure between the two syringes.

In some embodiments the two syringe fluid flow module 104 may be integrated with device 100, wherein intrinsic electronics module 110 and sensor module 120 are utilized to operate flow module 104. In some embodiments, the two syringe fluid flow module 104 may be configured as an external and/or auxiliary device 104e, a non limiting example of which is shown in FIG. 6, wherein electronics 104c and sensors, particularly pressure sensor 121, are configured to render module 104e operational as an independent and/or auxiliary unit to device 100 therein forming a system 150.

FIG. 6 shows an optional embodiments of device 100 and system 150 wherein an external and/or auxiliary fluid flow module 104 is provided labelled 104e. Flow module 104e may be provided in the form of a two syringe assembly and associated via introduction port 106i where a first syringe utilized, acting as a negative pressure sub-module 104a, utilized to aspirate and/or suction the tissue treatment site, and a second syringe utilized to act as a positive pressure sub-module 104b so as to introduce a fluid to the treatment site, for example a saline solution. Optionally and preferably auxiliary flow module 104e features a pressure sensor 121 and associated electronics and controller 104c, to monitor pressure values. In some embodiments pressure sensor 121 and controller 104c provide for measuring and equilibrating pressure between the two syringes 104a, 104b compartments. As shown each negative pressure sub-module 104a and positive pressure sub-module 104b may be directly associated and in fluid communication with a working flow channel 106a, 106b via port 106i, optionally via catheter or the like extending from each flow channel 106a,b respectively.

While the invention has been described with respect to a limited number of embodiment, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not described to limit the invention to the exact construction and operation shown and described and accordingly, all suitable modifications and equivalents may be resorted to, falling within the scope of the invention.

It should be noted that where reference numerals appear in the claims, such numerals are included solely or the purpose of improving the intelligibility of the claims and are no way limiting on the scope of the claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

While the invention has been described with respect to a limited number of embodiments, it will be appreciated that many variations, modifications and other applications of the invention may be made.

1. A tissue ablation device, the device comprising, a body, a working channel module featuring at least one working channel (106a), fluid flow module, an electronics module, a sensor module, and an expandible ablation member that is functionally coupled to an actuator.

2. The tissue ablation device of claim 1 wherein said ablation member and actuator are coupled so as to allow said ablation member to rotate.

3. The tissue ablation device of claim 1 wherein said actuator is a piezoelectric actuator.

4. The tissue ablation device of claim 1 wherein said actuator is a motor.

5. The tissue ablation device of claim 1 wherein said expandible ablation member is a stent like member having a controllable three dimensional shape.

6. The tissue ablation device of claim 1 wherein the shape and/or at least one dimension of said expandible ablation member is controllable.

7. The tissue ablation device of claim 6 wherein the shape and/or at least one dimension of said expandible ablation member is controllable based on at least one parameter of the tissue treatment site.

8. The tissue ablation device of claim 7 wherein said at least one tissue treatment site parameter is selected from: length, width, height, area, volume, shape, surface area, tissue type, or any combination thereof.

9. The tissue ablation device of claim 1 wherein said at least one working channel (106,106a) is configured to provide for aspiration.

10. The tissue ablation device of claim 1 wherein said at least one working channel is configured to provide for delivery of a flowing fluid.

11. The tissue ablation device of claim 1 wherein said sensor module comprises an image sensor configured to determine at least one tissue ablation site parameter.

12. The tissue ablation device of claim 1 wherein said fluid flow module is configured to functionally associate with said working channel module.

13. The tissue ablation device of claim 12 wherein said fluid flow module comprises a negative pressure module configured to generate suction in at least one working channel of said working channel module.

14. The tissue ablation device of claim 12 wherein said fluid flow module comprises a positive pressure module configured for delivering a flowing fluid to a tissue treatment site.

15. The tissue ablation device of claim 12 wherein said fluid flow module is provided in the form of a syringe.

16. The tissue ablation device of claim 1 wherein said body comprises a distal portion and a proximal portion.

17. The tissue ablation device of claim 16 wherein the length of distal portion (102d) is controllable.

18. The tissue ablation device of claim 16 wherein at least a portion of said distal portion (102d) is maneuverable.

19. The tissue ablation device of claim 1 wherein said body comprises a working channel introduction port.

20. A tissue ablation system comprising the tissue ablation device of claim 1, an introducing module (10) and an auxiliary device that is functionally coupled with said tissue ablation device.

21. (canceled)
